(19) United States
(12) Patent Application Publication
(10) Pub. No.: US 2006/0258752 A1
(43) Pub. Date: Nov. 16, 2006

(54) Method and compounds for cancer treatment utilizing NFkB as a direct or ultimate target for small molecule inhibitors

(76) Inventors: David L. Vander Jagt, Albuquerque, NM (US); Dorraine M. Deck, Albuquerque, NM (US); Steve F. Abcouwer, Hummelstown, PA (US); Ekaterina Bobrovnikova-Marjon, Philadelphia, PA (US); Waylon M. Weber, Albuquerque, NM (US)

Correspondence Address: MUETING, RAASCH & GEBHARDT, P.A.;ATTENTION: VICTORIA A. SANDBERG, P.O. BOX 581415, MINNEAPOLIS, MN, 55458-1415, US

(21) Appl. No.: 11/057,736

(22) Filed: Feb. 14, 2005

Related U.S. Application Data
(60) Provisional application No. 60/544,424, filed on Feb. 12, 2004.

(51) Int. Cl. A61K 31/12

(52) U.S. Cl. 514/688

(57) ABSTRACT

A method is described for cancer treatment through NFκB inhibition. NFκB is a direct or ultimate target for small molecule inhibitors. These small molecule inhibitors are aimed at suppression of NFκB directly or by indirect suppression of IKK, SFK kinases, or other upstream kinases. The present invention includes small molecule inhibitors comprising three, five, and seven carbon unsaturated spacers having one or two carbonyls, flanked by substituted aryl rings. The small molecule inhibitors can be symmetrical or unsymmetrical.

### FIELD OF THE INVENTION

The present invention pertains generally to assistive treatment of cancer by suppression of NFκB expression either directly or indirectly. The present invention is particularly, but not exclusively, useful for improving the effectiveness of chemotherapeutic agents by preventing NFκB's promotion of factors responsible for angiogenesis and metastasis. Various small molecule inhibitors may be utilized for direct or ultimate NFκB suppression.

### BACKGROUND OF THE INVENTION

NFκB was first identified as the nuclear factor in mature B-lymphocytes that binds to an 11 bp element (GGGACTTTCC) within the κ-light chain gene enhancer, but it was soon realized that NFκB is not a B-cell-specific transcription factor. A wide variety of environmental stimuli and stresses lead to the formation of active NFκB complexes within almost every cell type, and NFκB activation mediates the transcription of over 180 target genes.

NFκB complexes are heterodimeric molecules composed of members from each of two NFκB functional groups: (1) NFκB1/p50 and NFκB2/p52, and (2) RelA/p65, RelB, and RelC (The most prevalent active complex is composed of NFκB1/p50 and RelA/p65 subunits). All subunits contain conserved 300 amino acid portions known as “rel homology domains” that contain nuclear location signals (NLS). Under “non-stimulated” conditions, NFκB is kept inactive by the restriction of these subunits to the cytoplasm. Activation of NFκB-responsive genes requires the exposure of NLS and translocation of the complex into the nucleus. The NLS of NFκB functional group members, RelA, RelB, and RelC, are blocked by binding to ankyrin repeat domain-containing proteins (the so-called inhibitors of NFκB): IκBα, IκBβ, IκBγ, and IκBε. NFκB is typically retained in the cytoplasm by binding to an IκBα protein. Activation is dependent upon the nuclear localization of the complex following its release from IκBα, where release of NFκB is stimulated through phosphorylation of serine residues located in the N-terminal protion of IκBα. Release is accomplished as serine phosphorylation leads to binding of IκBα by β-TrCP, ubiquination by an E3 ubiquitin ligase complex (SCF composed of Skp-1, Cul-1, and Roc1), and degradation of lκB by the 26 S proteosome. IκB is phosphorylated on serines by enzyme complexes known as IκB kinases, composed of subunits IKKα (IKK1), IKKβ (IKK2), or IKKγ (NEMO, IKKAP). IKK is also activated by phosphorylation, for example, by the NFκB-inducing kinase (NIK). Phosphorylation and activation of IKK seems to result from the stimulation of several signal transduction kinase cascades.

Recently it has become apparent that the above paradigm is not strictly true for inhibition of NFκB function by IκBα. Whereas binding to IκBβ effectively sequesters NFκB in the cytoplasm, binding to IκBα does not preclude nuclear translocation. In fact, the NFκB-IκBα trimeric complexes shuttle between the cytoplasm and the nucleus. The source of this difference is that binding of IκBβ to a p50/p65 complex blocks NLS located on both NFκB subunits, whereas binding to IκBα blocks only the p65 NLS. Thus, NFκB-IκBα complexes contain both an exposed functional NLS and several nuclear export signals (NES) found in the N-terminal domain of IκBα and in the activation domain of p65. The functions of both NLS and NES result in this shuttling between the cytoplasm and the nucleus. However, multiple NES seem to dominate, resulting in a primarily cytoplasmic localization of NFκB-IκBα complexes. When nuclear export is blocked with leptomycin B (LMB), the complex accumulates in the nucleus. Since IκBα is the most prevalent IκB isoform, in most resting cells the majority of NFκB protein is located in the cytoplasm bound to IκBα. Inflammatory stimuli, such as IL-1 treatment, leads to activation of IKK activity, phosphorylation of IκBα on serine 32 and 36, recognition of IκBα by the E3 ubiquitin ligase, IκBα ubiquination, degradation of IκBα by the 26 S proteasome, and release of NFκB. The two exposed NLS on NFκB subunits then cause nuclear translocation of the transcription complex. However, numerous studies have now documented states where NFκB activation occurs in the absence of IκBα degradation.

The transcription factor NFκB, which is well known for its role in inflammatory diseases, is now also known to play a key role in cancer. NFκB is active in many tumors, and expression of NFκB-responsive genes provide cancer cells with distinct survival advantages that inhibit cancer treatment. NFκB is constitutively activated in many cancer cells, and NFκB may also be conditionally activated in both cancer cells and stromal cells by the tumor microenvironment. Normally, NFκB activation is prevented by binding to inhibitor (IκB) proteins, the most prevalent being inhibitor of NFκB alpha (IκBα). In response to inflammatory cytokines, the release of NFκB is triggered by phosphorylation of IκBα on serines 32 and 36, resulting in ubiquination and degredation of IκBα protein. However, in cancer cells subjected to environmental conditions such as hypoxia or X-rays, NFκB activation is caused by phosphorylation of IκBα on a tyrosine residue (Tyr42) by Src family kinases (SFKs). We hypothesize that this mechanism also leads to activation of NFκB in response to nutrient starvation. Thus, NFκB activation via IκBα Tyr42 phosphorylation is expected to occur in solid tumors due to constitutive activation of SFKs such as the Src oncogene in response to the hypoxic and nutrient poor nature of the tumor microenvironment, or due to radiation treatment of the tumor. Because NFκB responsive genes can promote angiogenesis, cell motility and invasion, and block apoptotic cell death, this mechanism represents a considerable obstacle to cancer treatment. Therefore, there is a greatly felt need for development of small molecule inhibitors of NFκB expression. Particularly, but not exclusively, inhibitors of IκBα Tyr42 phosphorylation have vast potential to serve as adjuvant cancer therapeutics.

The evidence that links activation of NFκB to oncogenesis is compelling: (1) NFκB is activated by a number of viral transforming proteins; (2) inhibition of NFκB activation through expression of a dominant negative IKK can block cell transformation; (3) NFκB activation protects cells from apoptosis induced by cancer chemotherapeutics and oncogenes; (4) NFκB activation results in up-regulation of cyclin D1, a cell cycle regulator that is up-regulated in many tumors; (5) activation of NFκB promotes expression of metastatic factors; (6) NFκB is constitutively expressed in many cancer cell lines; (7) a number of dietary chemopreventive compounds such as flavonoids, curcumin and resveratrol block activation of NFκB; and (8) the expression of interleukin-8 (IL-8) which has been identified as a key factor in both angiogenesis and metastasis, is very dependent on NFκB activity.

As discussed above, there are five members in the NFκB family, distinguished by the presence of a Rel homology domain. Each NFκB member is retained in the cytosol as a complex, the most prevalent of which is a dimer consisting of the p65 and p50 subunits. However, also in the cytosol is a set of proteins, designated IκB, that inhibit NFκB. Phosphorylation of IκB by IκB kinase (IKK) in response to an array of signals leads to the undesired degradation of IκB and the release of NFκB in the context of cancer treatment. This free NFκB is tranlocated to the nucleus where it binds to promoter regions of DNA resulting in the activation of a battery of genes, including anti-apoptotic pro-survival genes. Therefore, given the mechanisms of suppression and expression of NFκB, compounds inhibiting the activation of NFκB can be directed at IKK, SFK, or other kinases at NFκB-DNA interactions. Kinase inhibitors will prevent phosphorylation of IκB whereas direct inhibitors of NFκB may block NFκB-DNA interactions, as shown in FIG. 7.

There are really two IKK's, designated IKKα and IKKβ, that exist in a complex called the IKK signalsome. Also included in the complex are the IKK-associated protein (IKAP) and NEMO (also called IKKγ). There are many upstream regulators of the IKK signalsome that have been identified and could be useful “targets” for suppression of IKK expression and, ultimately, NFκB expression. Thus, compounds that prevent the phosphorylation of IκB (and therefore prevent the activation of NFκB) may accomplish prevention of expression of NFκB by acting directly on one or more members of the IKK signalsome or by inhibiting upstream kinases, such as SFK or any other such family of kinases. This complicates simple structure-based design of potential drugs to prevent activation of NFκB, especially because crystal structures of the IKK signalsome are not available in the art presently. It is noteworthy that there are also IKK-independent pathways for activation of NFκB.

NFκB crystal structures are available for use in structure-based drug design including a human NFκB-DNA structure. However, compounds that have been reported to inhibit activation of NFκB have generally been suggested or demonstrated to work at the level of IKK, rather than to interfere with NFκB-DNA interactions or with NFκB dimerization to prevent its interactions with DNA. For example, it has been shown recently that a new class of retinoid-related anticancer agents inhibits IKK directly. Likewise, a synthetic derivative of the fungal metabolite jesterone, which blocks activation of NFκB, was shown to specifically inhibit IKKβ. It appears, therefore, that inhibition of IKK (or SKK or other kinases) may be a promising route to the development of anticancer agents that work by promoting apoptosis through blocking the activation of NFκB at an upstream kinase level.

For centuries, curcumin has been used in India and southeast Asia as a medicinal for a wide variety of conditions such as internal and external wounds, hepatitis, bile duct disorders, and rheumatoid arthritis. Curcumin has been reported to possess antioxidant, anti-inflammatory, antiviral, and antimutagenetic activities. It has also been shown to possess anticancer properties. Curcumin is a natural chemoprotective agent that elevates the activities of Phase 2 detoxification enzymes, while inhibiting procarcinogen activating Phase 1 enzymes. It decreases expression of several proto-oncogenes including c-jun, c-fos, and c-myc, and of particular interest, it suppresses the activation of NFκB. Related to this, curcumin has also been shown to induce apoptosis in several tumor cell lines. In addition to the down-regulation of uPA by dominant negative inhibitors of NFκB, numerous other factors, including VEGF, IL-8, and MMP-9 that contribute to angiogenesis, invasion, and metastasis are down-regulated by dominant negative inhibitors of NFκB. Likewise, curcumin inhibits angiogenesis in vivo. Curcumin can be viewed as a lead compound that inhibits metastasis and promotes apoptosis. Therefore, development of inhibitors of activation of NFκB as potential new therapeutics to prevent metastasis by examining analogs of curcumin was undertaken in order to provide small molecules inhibitors for adjuvant therapeutic agents for treatment of cancer and to examine treatment of cancer by inhibiting activation/expression of NFκB.

The advantages, objects and features of such a treatment route and treatment pharmaceuticals will become apparent to those skilled in the art when read in conjunction with the accompanying following description, drawing figures, and appended claims.

As those skilled in the art will appreciate, the conception on which this disclosure is based readily may be used as a basis for designing other structures, methods, and systems for carrying out the purposes of the present invention. The claims, therefore, include such equivalent constructions to the extent the equivalent constructions do not depart from the spirit and scope of the present invention. Further, the abstract associated with this disclosure is neither intended to define the invention, which is measured by the claims, nor intended to be limiting as to the scope of the invention in any way.

### SUMMARY OF THE INVENTION

The present invention is a method for treatment of cancer in mammals by suppression of NFκB expression by providing a therapeutically effective amount of a curcumin derivative and administering the curcumin derivative to the mammal, using a pharmaceutically acceptable carrier. The method of administering the treatment is by a method of administration selected from oral administration, parenteral administration, transcutaneous administration, intranasal administration, intramuscular administration and rectal administration. The suppression of NFκB is direct suppression. The suppression of NFκB is indirect suppression by at suppression of at least one of IKK, SFK kinases, other upstream kinases.

### BRIEF DESCRIPTION OF THE DRAWING

FIG. 1 is a formulaic chemical representation of polyphenolic curcumin.

FIG. 2 is a formulaic representation of a synthetic scheme utilizing an aldol reaction with acetylacetone and substituted benzaldehydes to form curcumin or its analogs; two analogs were synthesized by treatment with palladium on activated charcoal under a hydrogen atmosphere; two analogs were synthesized by treatment with a base and an alkyl halide;

FIG. 3 is a formulaic representation of a synthetic scheme utilizing a base catalyzed aldol reaction with acetone and substituted benzaldehydes; two analogs were also synthesized using a base catalyzed aldol reaction with excess acetone and substituted benzaldehydes two analogs were synthesized by treatment with palladium on activated charcoal under a hydrogen atmosphere;

FIG. 4 is a formulaic representation of a synthetic scheme utilizing analogs synthesized using a base catalyzed aldol reaction with substituted acetophenones and substituted benzaldehydes;

FIG. 5 is an activity chart depicting activity of tested small molecules synthesized according to the schemes set forth in FIGS. 2-4 in relation to curcumin in reducing expression of NFκB;

FIG. 6 is a chemical formula representation of the four preferred general compounds of the invention;

FIG. 7 is a representative drawing of the activation/inhibition pattern of NFκB in relation to the structure of the molecule, activity by IκB, and NFκB's positioning in a cell; and

FIG. 8 is a listing of some derivatives that are particularly useful for the treatment described.

### DESCRIPTION OF A PREFERRED EMBODIMENT

The present invention comprises treatment of cancer in humans and other mammals, as described more fully hereinafter. This invention, may, however, be embodied in different forms and is not limited to the embodiments set forth herein, but the embodiments are set forth only to ensure that those skilled in the art will be enabled in applying the invention.

The terminology as set forth herein is for description of the embodiments only and should not be construed as limiting of the invention as a whole. As used in the description of the invention and the appended claims, the singular forms “a”, “an”, and “the” are inclusive of their plural forms, unless contraindicated by the context surrounding such.

All technical and scientific terms used herein have the commonly understood meaning of one skilled in the art. All publications, patent applications, patents and other references disclosed herein are incorporated by reference in their entirety.

The term “alkyl” or “lower alkyl” refers to C1 to C8 alkyl, which may be linear, branched, saturated, and/or unsaturated.

The term “cycloalkyl” typically refers to C3-C8 cycloalkyl.

The term “alkenyl” or “lower alkenyl” as used herein refers to C1 to C4 alkenyl.

The term “alkoxy” or “lower alkoxy” refers to C1 to C4 alkoxy.

The term “aryl” refers to C3 to C10 cyclic aromatic groups such as phenyl, naphthyl, and the like, and includes substituted aryl groups such as tolyl.

“Halo” refers to any halogen group such as chloro, fluoro, bromo, or iodo groups.

“Hydroxyalkyl” as used herein refers to C1 to C4 linear or branched hydroxyl-substituted alkyl, for example, —(CH₂)₂OH.

The term “aminoalkyl” refers to C1 to C4 linear or branched amino-substituted alkyl, wherein “amino ” refers to the group NR′R″, wherein R′ and R″ are independently selected from H or lower alkyl as defined above, for example, —NH₂, —NHCH₃, —N(CH₃)₂.

The term “oxyalkyl” as used herein refers to C1 to C4 oxygen-substituted alkyl, i.e., —OCH₃, and the term “oxyaryl” refers to C3 to C10 oxygensubstituted cyclic aromatic groups.

The term alkylenedioxy” refers to a group of the general formula —OR′O—, —OR′OR′—, or R′OR′OR′— where each R′ is independently alkyl.

“Treat”, “treating”, “treatment”, etc. as used herein refer to any action providing a benefit to a patient afflicted with a disease, including improvement in the condition through lessening or suppression of at least one symptom, delay in progression of the disease, prevention or delay in the onset of the disease, etc.

“Pharmaceutically acceptable” as used herein means that the compound or composition is suitable for administration to a subject to achieve the treatments described herein, without unduly deleterious side effects in light of the severity of the disease and necessity of the treatment.

“Inhibit” as used herein means that a potential effect is partially or completely eliminated.

The present invention is concerned primarily with the treatment of human subjects, but may also be employed for the treatment of other animal subjects (i.e., mammals, avians) for veterinary purposes. Mammals are preferred, with humans being particularly preferred.

The transcription factor nuclear factor κB (NFκB) is well known as a regulator of genes controlling the immune and inflammatory responses. However, activation of NFκB is also associated with many aspects of oncogenesis, including control of apoptosis, differentiation, and cell migration. Thus NFκB can be viewed as a pro-survival signal. The overexpression of NFκB, which is observed in many tumors, can blunt the effectiveness of chemotherapy by promoting the pro-survival, anti-apoptotic state. Of particular interest is the role that NFκB may play in metastasis. Retroviral delivery of a dominant negative inhibitor of NFκB has been shown to down-regulate expression of a number of prometastatic factors including urokinase-type plasminogen activator (uPA). The serine protease uPA and its receptor uPAR are overexpressed in many tumors and are well-established participants in the metastatic process. Therefore, the observation that dominant negative inhibition of NFκB down-regulates the expression of uPA suggests that selective inhibitors of NFκB may be potential anti-metastatic therapeutics, and that suppression of NFκB may be a viable treatment route against cancer.

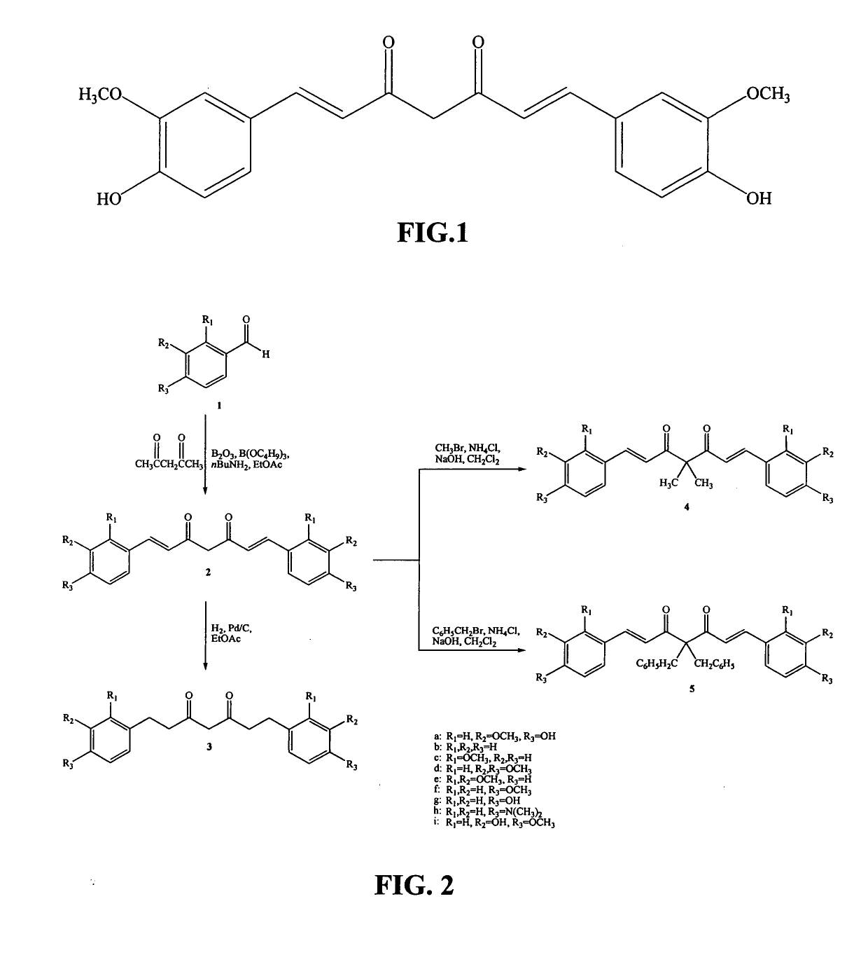

Polyphenolic curcumin, as depicted in FIG. 1 has been used for centuries as an antioxidant and food preservative in its natural form in the spice turmeric. It has more recently been found to prevent activation of NFκB and to generally exhibit “anti-cancer” activity. Therefore, analogs of this compound may also exhibit similar and perhaps even greater activity.

Curcumin (FIG. 1), is a symmetrical molecule containing two aryl rings separated by a conjugated unsaturated seven carbon spacer having two carbonyls. The aryl rings of curcumin contain a hydroxyl group in the para position and a methoxy group in the meta position.

Synthesis of Analogs

Several analogs were synthesized that have some structural similarity to curcumin.

7-C Spacers

The first series of compounds contained two aryl rings separated by an unsaturated seven carbon spacer having two carbonyls. The aryl rings contained different substituents in various positions on the ring, wherein the structure of the substituents was designed to test the importance of the type of functional group and location on the aryl ring necessary for inhibition. The synthesis of compounds 2a-2i was performed using an aldol type reaction as first described by Pabon (Pabon, H. J. J. 1964. A Synthesis of Curcumin and Related Compounds. Recueil 379-386). 2,4-Pentanedione was reacted with a substituted benzaldehyde (FIG. 2) to give curcumin (2a) or one of its analogs (2b-2i).

Two curcumin analogs, 3a and 3b were synthesized from analogs 2a and 2b according to the scheme found in FIG. 2, using palladium on activated charcoal under a hydrogen atmosphere. These compounds contain two aryl rings separated by a saturated seven carbon spacer having two carbonyls and were designed to test the importance of having saturation in the seven carbon spacer.

Four additional curcumin analogs, 4b, 5b, 6b, and 7b were synthesized from analog 2b according to the scheme found in FIG. 2. These contain two aryl rings separated by an unsaturated seven carbon spacer having at least one substituent between the carbonyls. These two curcumin analogs were designed to test the importance of substituents on the spacer and on the central methylene carbon. The synthesis of compounds 4b and 5b was performed by addition of a base and an alkyl halide in an S_{N}2 type reaction. The disubstituted compound 5b was formed rather than the monosubstituted compound 7b. Compounds 6b and 7b were prepared, respectively, by reacting 3-methyl-2,4-pentanedione and 3-benzyl-2,4-pentanedione with benzaldehydes in an aldol type reaction.

5-C Spacers

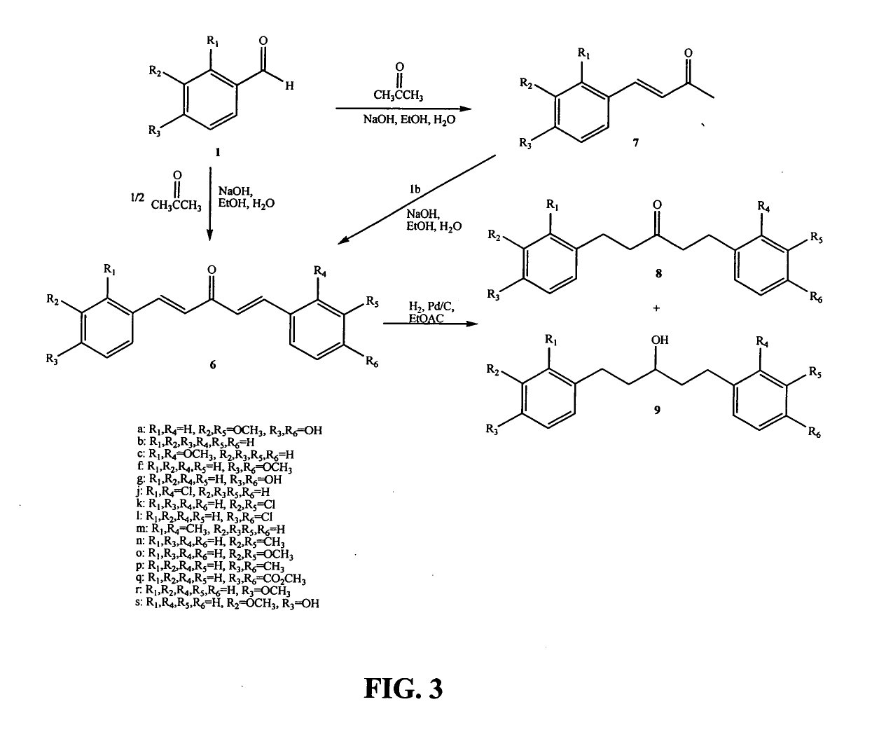

A second series of compounds 6a-6c, 6f, 6g, 6j-6q were synthesized containing two aryl rings separated by a five carbon unsaturated spacer having a single carbonyl. These compounds were designed to test the importance the length of the spacer and the number of carbonyls in the spacer. The synthesis of compounds 6a-6c, 6f, 6g, 6j-6q involves a base catalyzed aldol reaction with acetone and substituted benzaldehydes as depicted in FIG. 3.

Two additional compounds, 6r and 6s, having a five carbon spacer contain two different aryl rings. These compounds were designed to test the importance of symmetry in compounds with a five carbon spacer. As depicted in FIG. 3, these compounds were synthesized using consecutive base catalyzed aldol reactions as described by Masuda (Masuda, T., Jitoe, A., Isobe. J., Nakatani, N., Yonemori, S. 1993. Anti-Oxidative and Anti-inflammatory Curcumin-Related Phenolics from Rhizomes of Curcuma Domestica. 32:1557-1560).

Two additional compounds 7a and 7f contain a single aryl ring and a 4-carbon unsaturated chain with a carbonyl These compounds were designed to test the importance of the necessity of two aryl rings. Compounds 7a and 7f were synthesized as depicted in FIG. 3 using a base catalyzed aldol reaction with excess acetone and substituted benzaldehydes.

Two compounds, 8b and 9b, were synthesized as shown in FIG. 3. These compounds contain a saturated five carbon spacer. Compound 9b has a hydroxyl group on the spacer rather than a carbonyl. These compounds were designed to test the importance of unsaturation and the necessity of a carbonyl in the spacer. The synthesis of these compounds was performed by reacting compound 6b with palladium on activated charcoal under a hydrogen atmosphere to give a mixture of compounds 8b and 9b, which were separated by chromatography.

Compounds 9a-9v contain two identical aryl rings separated by an unsaturated five carbon spacer having a single carbonyl whereas compounds 9x and 9y have two different aryl rings. These compounds were designed to test the importance of the length of the spacer between the two aryl rings. Compounds 9a-9w were prepared from acetone and a substituted benzaldehyde in a base catalyzed aldol reaction as described by Masuda. In the case of phenolic benzaldehydes, the phenol was protected with a methoxymethyl group prior to the aldol reaction and deprotected later to give the free phenol. Compounds 9u and 9v were prepared from 9a and 9r respectively by reaction with acetic anhydride as described by Ali. Compounds 9x and 9y were prepared using two consecutive base catalyzed aldol reactions.

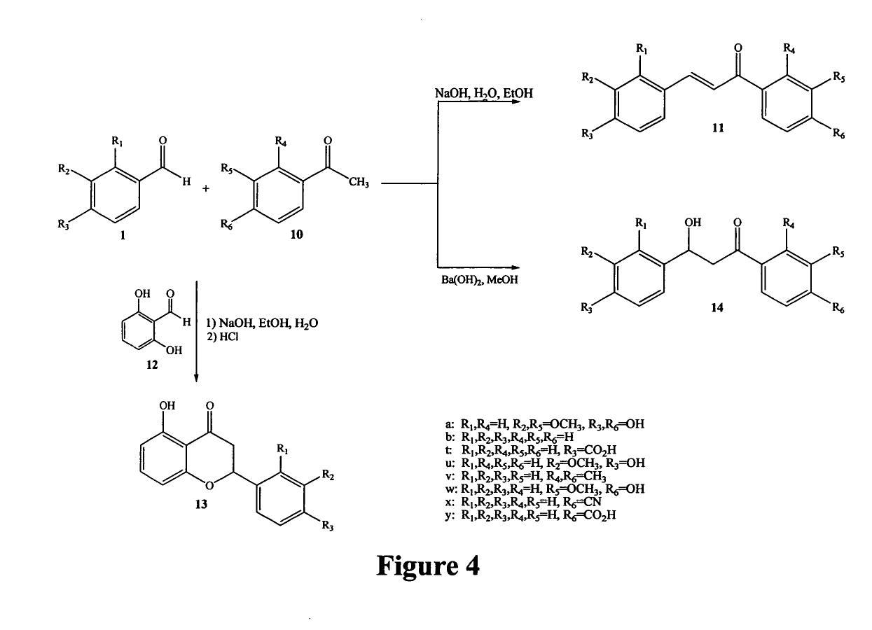

Compounds 8a, 8c and 8t were prepared as shown in FIG. 4. These compounds contain a single aryl ring with an unsaturated 3-carbon chain and a single carbonyl. These compounds were designed to test the necessity of two aryl rings. Compounds 8a, 8c and 8t were prepared from excess acetone and a substituted benzaldehyde in a base catalyzed aldol reaction following the procedure of Masuda.

Compounds 10b and 11b were prepared as shown in FIG. 4. These compounds, which have a saturated five carbon spacer, were designed to test the importance of unsaturation and the necessity of a carbonyl in the spacer of series 2 compounds. Compounds 10b and 11b were prepared by reduction of 9b.

Compounds 12a and 12b were prepared as shown in FIG. 4. These compounds contain two identical aryl rings separated by an unsaturated five carbon spacer having both a carbonyl and a saturated ring and were designed to test the importance of a ring in the spacer. They were synthesized following the procedure of Masuda by reaction of a substituted benzaldehyde with cyclohexanone in a base catalyzed aldol reaction.

Compound 13b was prepared as shown in FIG. 4. This compound contains two identical aryl rings separated by a five carbon spacer containing both a carbonyl and two epoxide rings. This compound was designed to test the importance of an epoxide on the spacer. Compound 13b was synthesized following the procedure of Yadav¹⁵ by reaction of 9b with t-butyl hydroperoxide.

3-C Spacers

Compounds, 11a, 11b, 11t-11y were synthesized as shown in FIG. 4. These compounds contain two aryl rings separated by an unsaturated three-carbon spacer having a single carbonyl. Six of these compounds are unsymmetrical having different substituents on the aryl rings. These compounds were designed to test the importance of the length of the spacer, the number of carbonyls, and symmetry in the molecule. The synthesis of these compounds was performed using a base catalyzed aldol reaction with substituted acetophenones and substituted benzaldehydes.

One compound 13b, was synthesized as shown in FIG. 4. Compound 13 contained no spacer and was designed to test the importance of a spacer. This compound was synthesized by a base catalyzed aldol reaction with a substituted acetophenone and a substituted benzaldehyde followed by an acid reaction.

One compound, 14u, was synthesized as shown in FIG. 4. Compound 14w contains a three-carbon spacer containing a carbonyl and a hydroxyl group. This compound was designed to test the importance of a hydroxyl group on the spacer. This compound was synthesized by using a base catalyzed aldol reaction with a substituted acetophenone and a substituted benzaldehyde.

All the structures in FIGS. 2-4 were verified by NMR and the known compounds were compared to literature data. This study allowed us to begin to develop versatile synthetic schemes for the preparation of curcumin analogs to test our hypothesis that modification of the curcumin structure would allow us to develop inhibitors of NFκB.

Analysis of Activity

The curcumin analogs synthesized as described above were compared to curcumin in a cell assay that employed HeLa cells transfected with a construct prepared using the BD Great EscAPe™ SEAP (Secreted Alkaline Phosphatase) Chemiluminescence kit, in which a promoter with multiple NFκB binding sites was cloned into SEAP. Transfection with this construct provided a cell line in which activation of NFκB by TNFα resulted in secretion of alkaline phosphatase, which was easily detected.

Details of the construct used for testing are as follows: The pNF-κB-SEAP-NPT plasmid that permits expression of the secretory alkaline phosphatase (SEAP) reporter gene in response to the NF-κB activation (contains SEAP cDNA under the control of thymidine kinase (TK) promoter and a 4×κB enhancer elements, GGGAATTTCC) and contains the neomycin phosphotransferase (NPT) gene for Geneticin resistance in host cells was kindly provided by Dr. Y. S. Kim (Moon K Y Hahn, B S, Lee J, Kim Y S. 2001 A cell based assay system for monitoring NF-kappa-B activity in human HaCat transfectant cells. Anal. Biochem. 292:17-21). HeLa cells were transfected with the NF-κB-SEAP-NPT vector as follows, Confluent HeLa cells (T175 flask) were trypsinized, resuspended in growth medium (DMEM supplemented with 4 mM glutamine, 100 units/ml penicillin, 100 μg/ml streptomycin, 0.25 μg/ml amphotericin B, and insulin), pelleted for 5 min at 1500 rpm, and resuspended again in 2 ml of RPMI 1640 media (no FBS). 1 ml of HeLa cells suspension (˜2×10⁷ cells) was mixed with 100 μg of NF-κB-SEAP-NPT vector DNA, placed into a cuvette and electroporated using Cell-Porator (Life Technologies™) at 1600 F and 200V. Afterward, electroporation cells were plated in T75 flask and allowed to recover for 24 h. Transfected cells were then transferred into 60 cm² dish and incubated in growth media supplemented with 6 mg/ml Genetecin (G418, Invitrogen). Stably transfected colonies were selected two to four weeks later using cloning cylinders. Clonal populations were screened for the ability to release SEAP into the culture media upon stimulation with 20 ng/ml TNF alpha for 24 h (R&D systems). Media samples (15 μl) were analyzed using the Great EscAPe SEAP chemiluminescence assay (Clontech).

Media:

DMEM (high glucose, no glutamine formulation) supplemented with 10% FBS (v/v), 4 mM glutamine, 100 units/ml penicillin, 100 μg/ml streptomycin, 0.25 μg/ml amphotericin B, insulin (1.1 ml of 10 mg/ml bovine insulin·Zn in 0.02 M HCl per liter of medium)+Genetecin (G418), 6 mg/ml final concentration (from Invitrogen, #11811-031).

Treatment:

To induce SEAP activity, confluent HeLa SEAP #15 was incubated with 20 ng/ml TNF alpha (R&D systems, #210-TA-010, rh TNF α) for 24 h. Basal or induced SEAP activity was inhibited by incubating with 50 μM curcumin in the presence or absence of 20 ng/ml TNF alpha for 24 h.

15 μl Media samples were collected. The concentration of SEAP in the media was high enough to be detected in the samples.

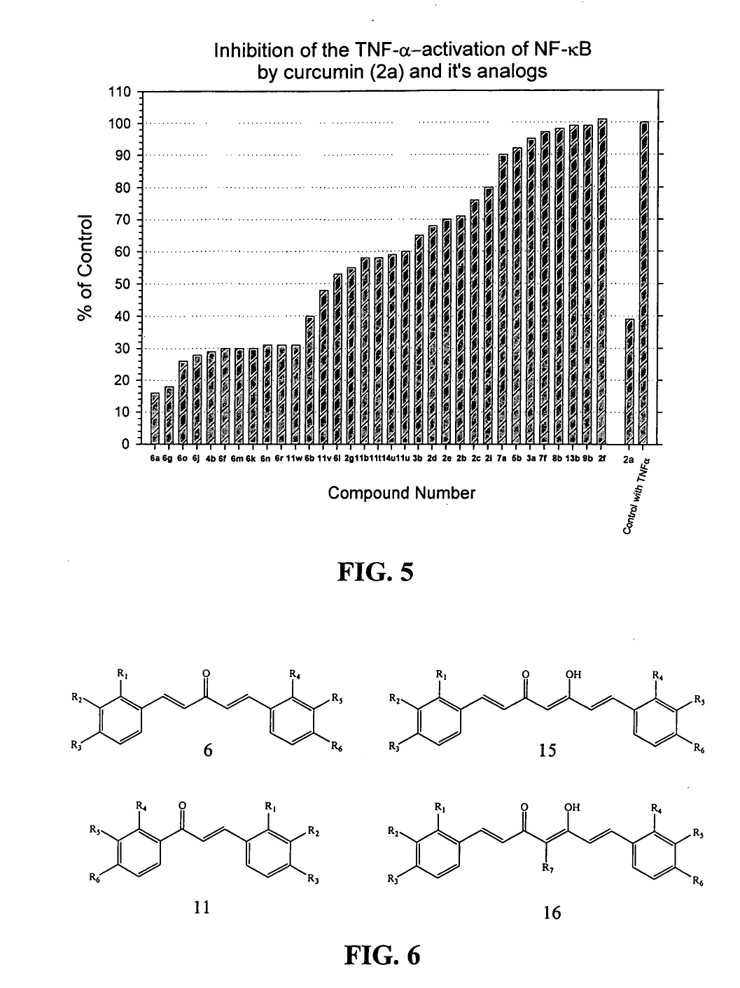

The activity of curcumin and its analogs was measured by their ability to decrease the level of secreted alkaline phosphatase, as shown in FIG. 5. Surprisingly, modest changes in structure produced marked alterations in activity, including producing analogs even more active than curcumin. In addition, some of the active analogs are quite far removed in structure from curcumin. In FIG. 5, the lower the bar, the greater the activity. Thus, analogs on the left are the most active.

Inhibition appears to be decreased when saturation is introduced into the linker segment of the analog. This could be due to a change in the geometry of the molecule. Inhibition also decreased when the analogs lacked a carbonyl in the linker or contained only one aryl ring. Hydroxy and methoxy substituents on the aryl rings added to inhibition. The hydroxyflavanone, compound 13b, was a poor inhibitior of NFκB.

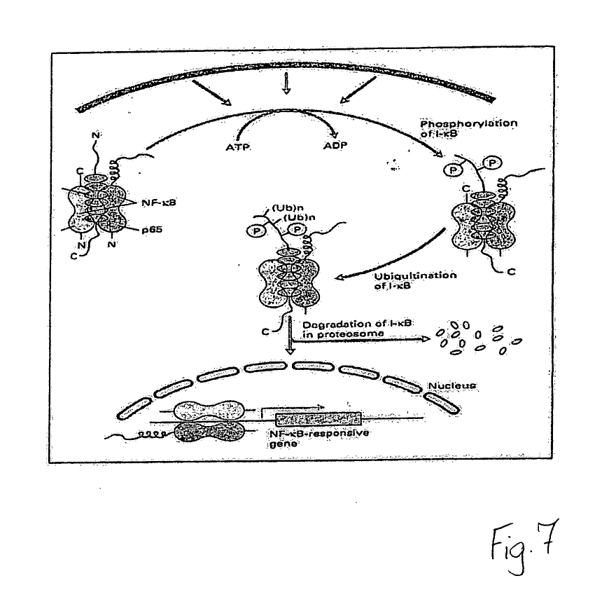

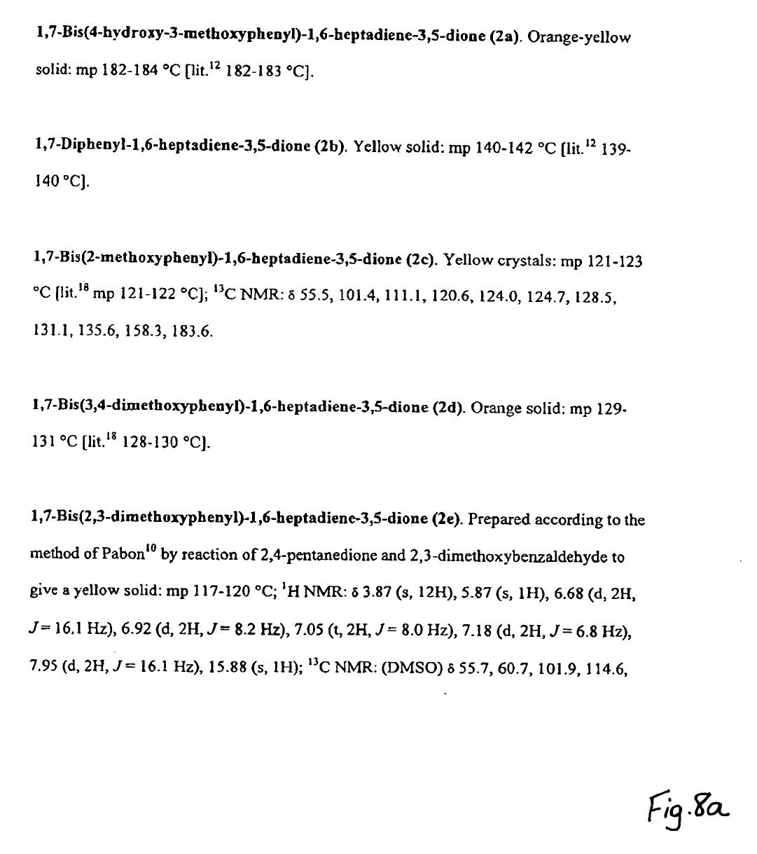

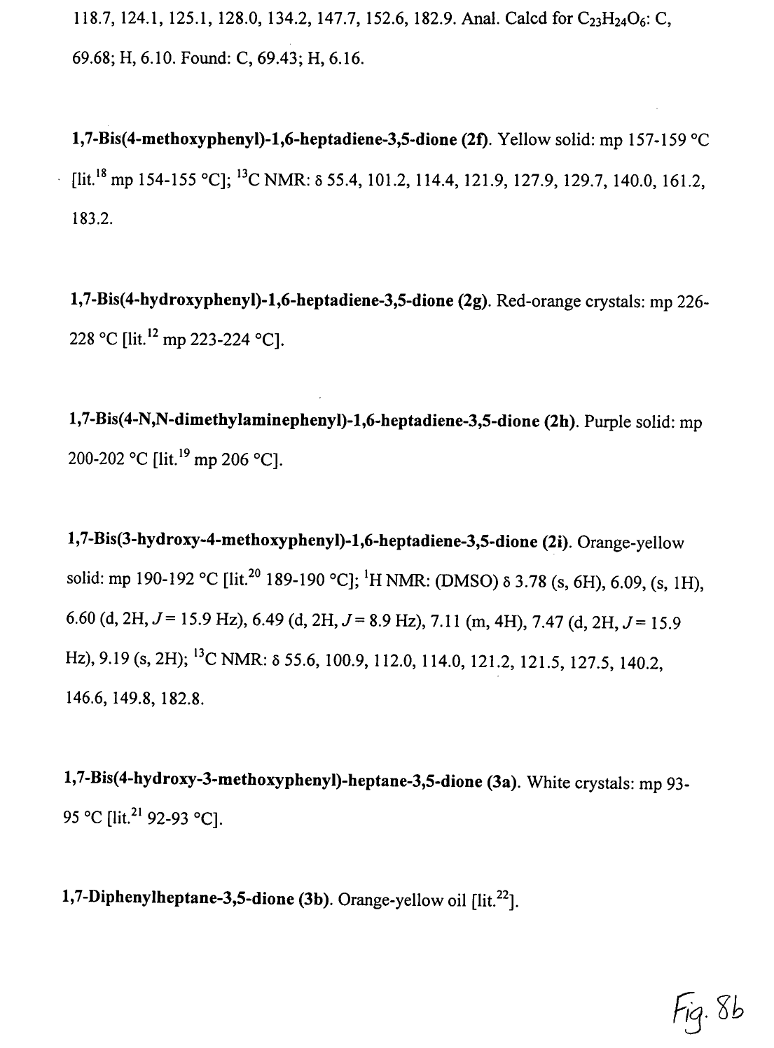

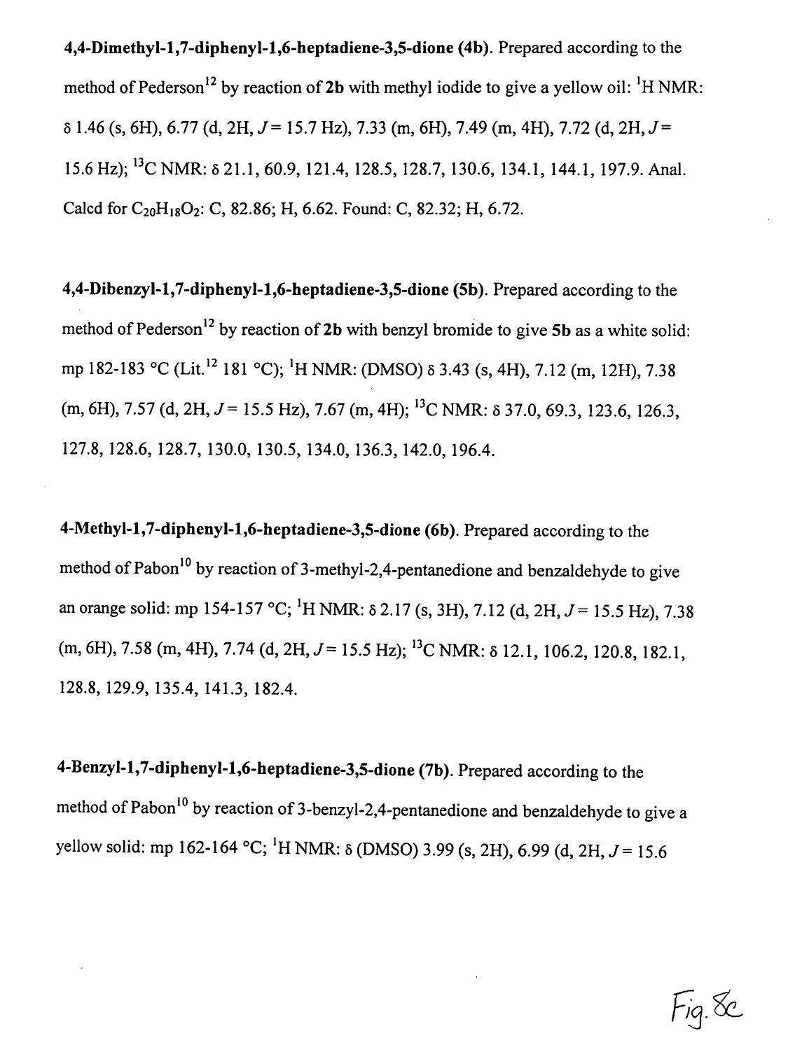

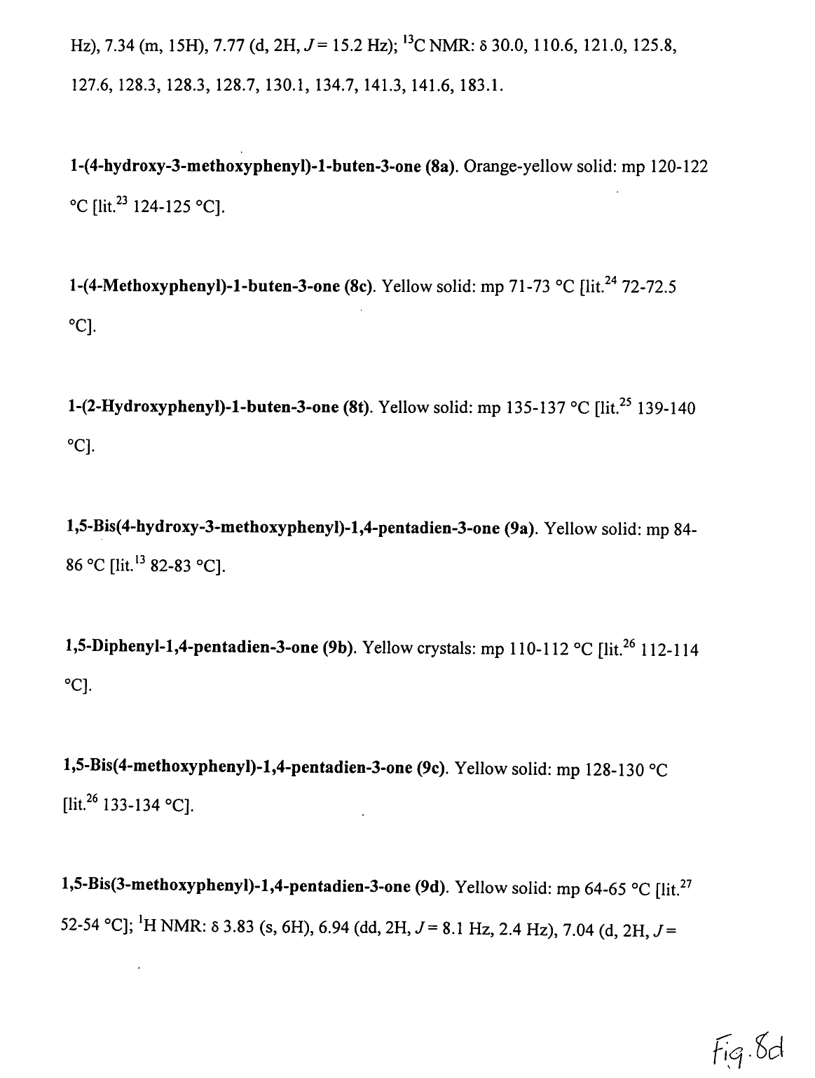

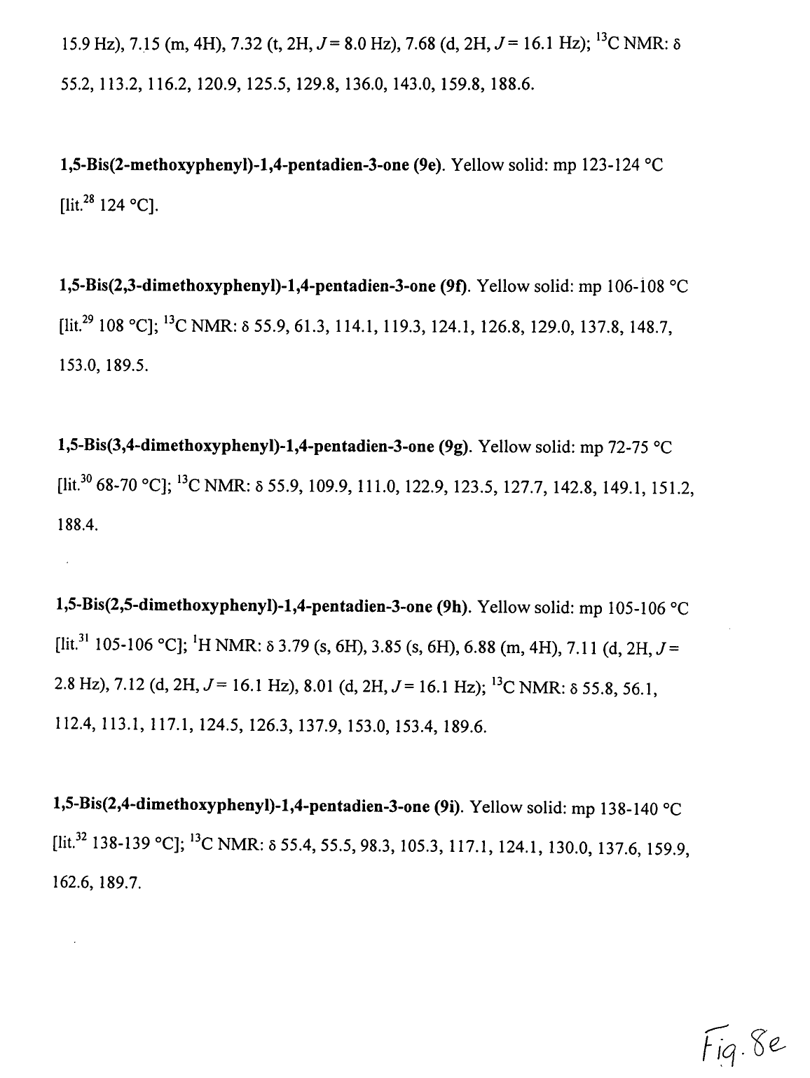

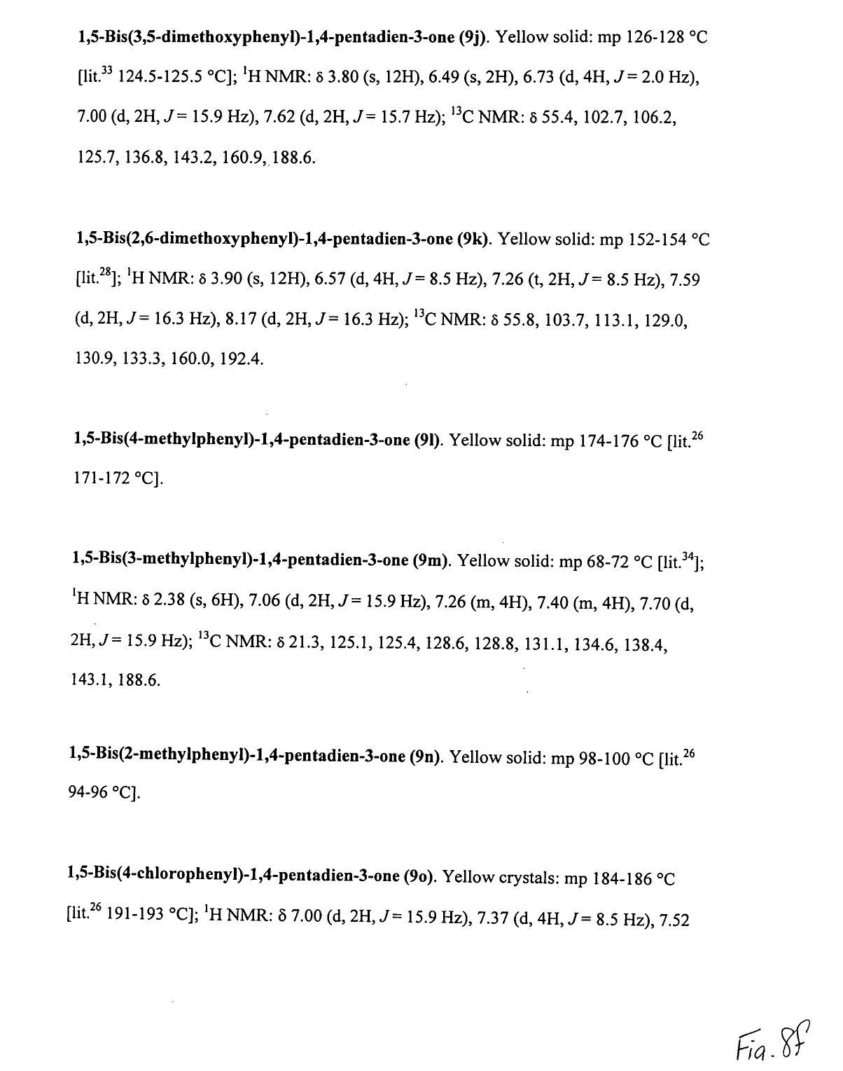

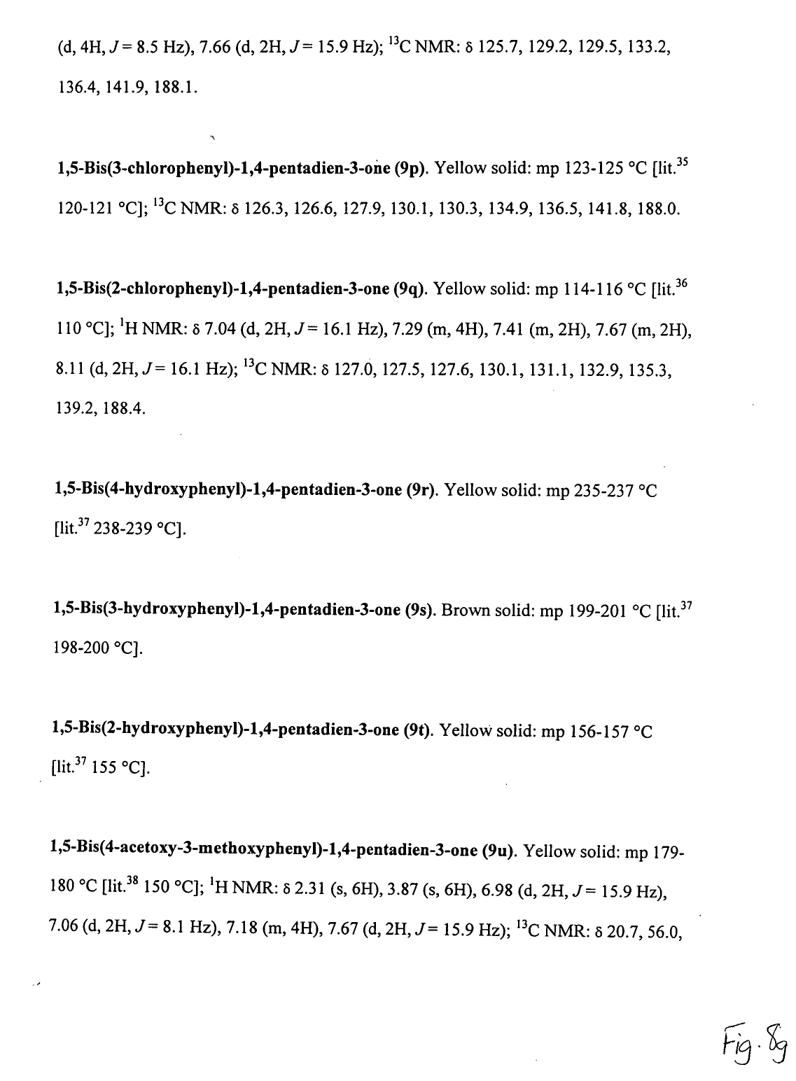

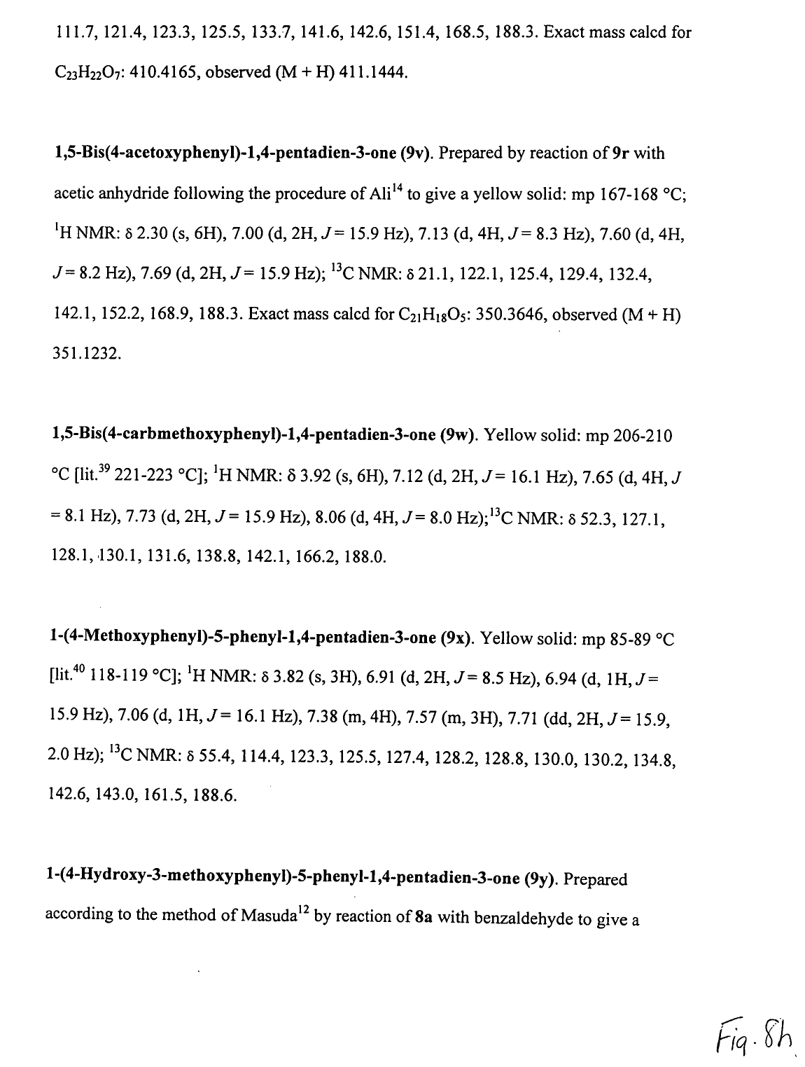

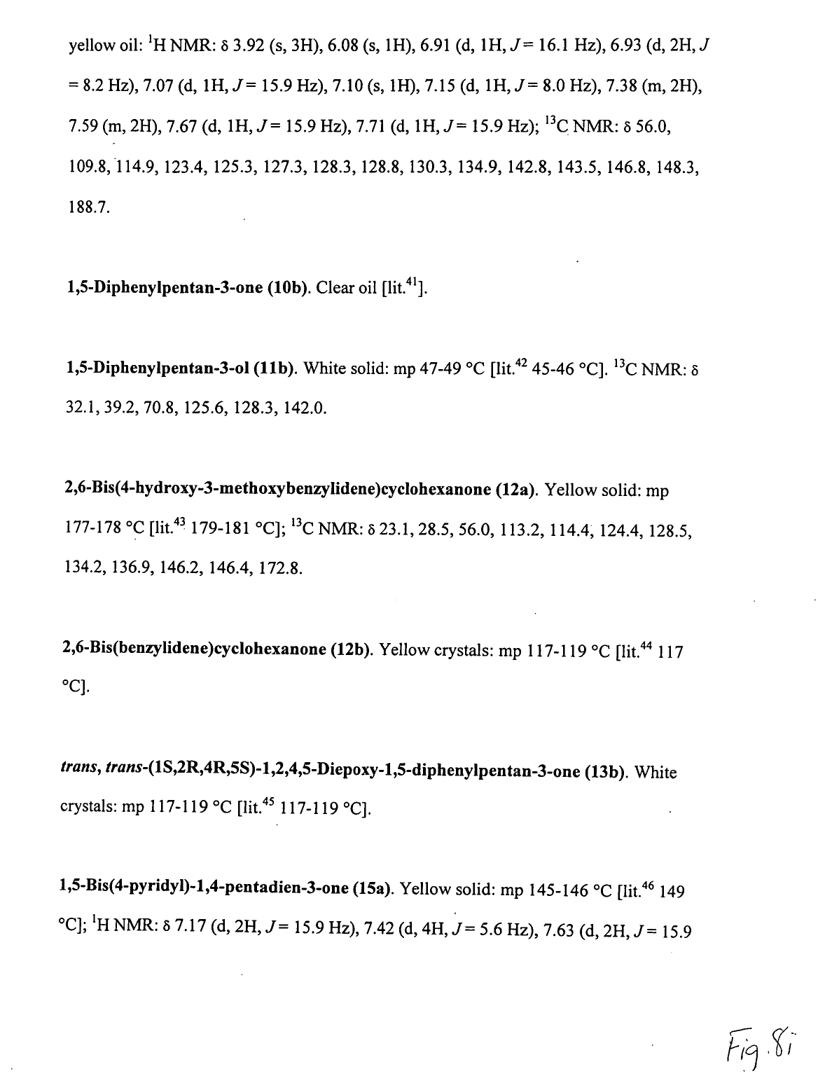

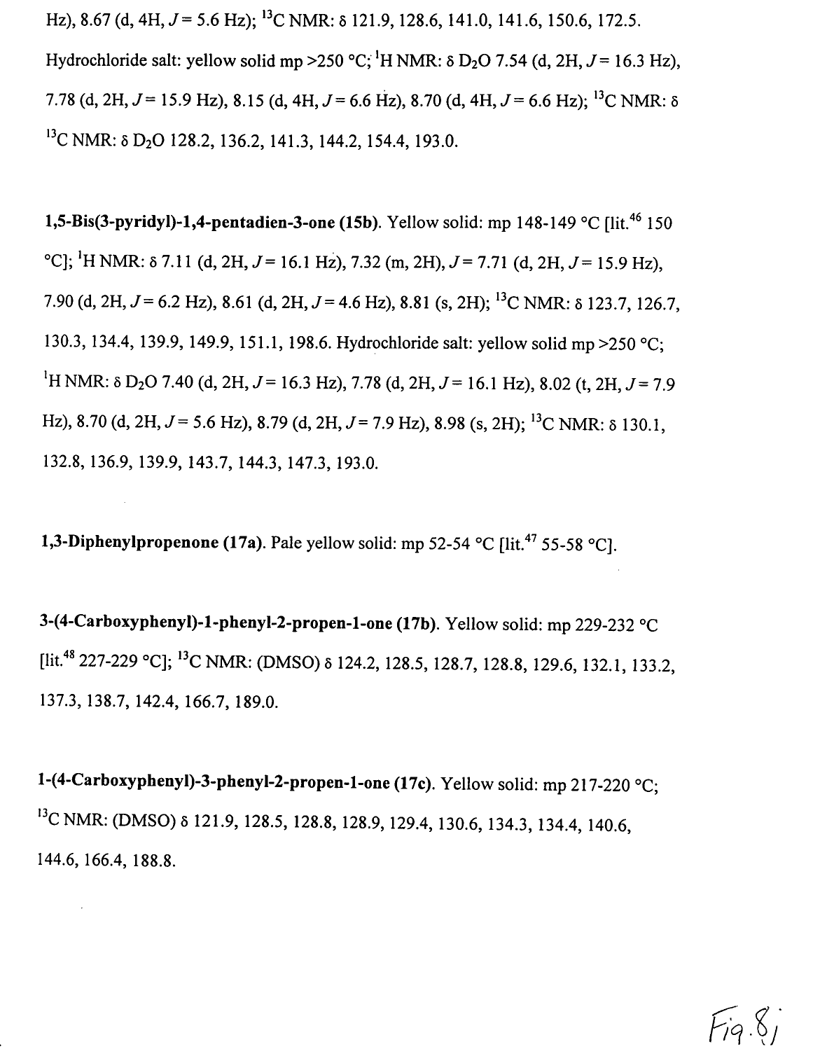

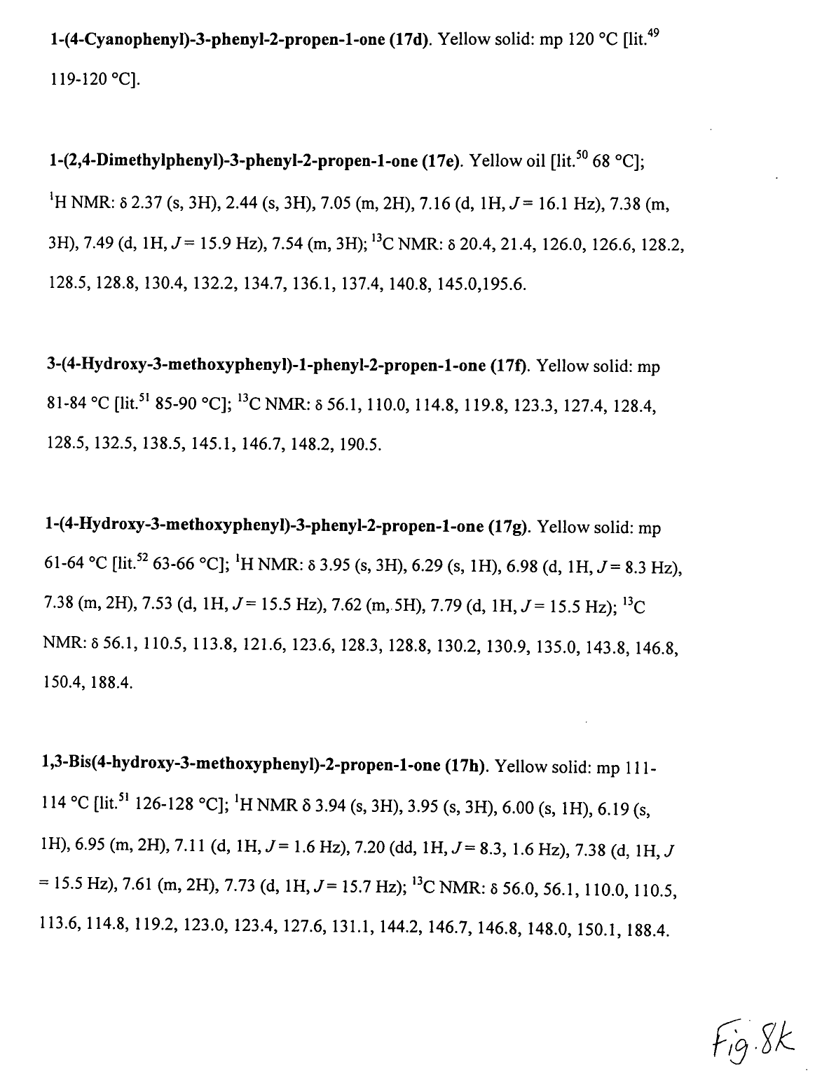

It was found that inhibition appeared to be highly dependent on the length of spacer, a carbonyl in the linker, and presence of substituents and their positions on two aryl rings. These compounds have seven, five, and three carbon spacers containing at least one alpha-beta unsaturated carbonyl. Various functional groups (alkyl, alkoxy, halo, etc.) in different positions on the rings alter activity, in some cases increasing activity. The substituents on the aryl rings may include hydroxyl and methoxy groups as well as halo, ester and carboxylic acid groups and any other substituent. The preferred general structures of the potential inhibitors are compounds 6, 11, and 15 (FIG. 6).

Initial inhibition data of these preferred potential inhibitor compounds having an unsaturated three-carbon linker indicate better inhibition if substituents are on the aryl ring closest to the carbonyl.

It is interesting to find that one potent inhibitor has a seven carbon spacer containing two alpha-beta unsaturated carbonyls and a methyl group on the methylene carbon of the spacer, as shown in FIG. 2 as in structure 4b. It is claimed that potentially any group can be added to this methylene carbon.

Curcumin analogs having the general structure 15 will preferably be synthesized using aldol chemistry. The appropriately substituted benzaldehydes will be reacted with a Masuda type modification to the Pabon method to afford analogs containing an unsaturated seven carbon spacer and aryl rings substituted with hydroxyl groups, esters and acids. Structures of analog 15 will be verified using NMR and analysis.

Analogs having a five carbon spacer and a single carbonyl as in compounds 6 will preferably be synthesized by reacting the appropriately substituted benzaldehydes with acetone as described in FIG. 3. The use of the chemistry described in FIG. 3 allows for the formation of symmetrical and unsymmetrical products. Verification of structure will be accomplished through NMR and analysis.

The shorter three carbon spacer analogs, having the structure 11, will preferably be synthesized as described in FIG. 4. Substituted acetophenones will be reacted with benzaldehydes to produce products having substituents on the aryl ring closest to the carbonyl.

Analogs having the structure 16 was preferably be synthesized by reacting compounds 2 or 15 with an alkyl halide as described in FIG. 2. Verification of the structures of compounds 11 and 16 was accomplished through NMR and analysis.

The aryl rings in any of the structures can be replaced by various heterocyclic rings to markedly increase the range of compounds claimed as potential inhibitors of NFκB.

The anti-oxidant activities of curcumin and analogues (Schemes 1-4) were determined in two standard assays. Antioxidant activity was measured as the ability of the analogues to react with the pre-formed radical monocation of 2,2′-azinobis-(3-ethylbenzothiazoline-6-sulfonic acid) (ABTS⁺). This assay is also known as the Total radical-trapping anti-oxidant parameter assay (TRAP assay). Anti-oxidant activity was also measured in the Ferric reducing/anti-oxidant power assay (FRAP assay) in which the compounds are reacted with ferric tripyridyltriazine complex. In both colorimetric assays, the vitamin E analogue Trolox was used as a control.

The results of the TRAP assay of anti-oxidant activity are shown in FIG. 2. There were active compounds in all three series. Generally, activity was observed with analogues that retained a phenolic substituent. In series 1, this included 3a, which is the reduced form of curcumin (2a) in which both of the enone double bonds have been reduced. Analogue 3a was the most active compound in the TRAP assay. Clearly, it is not necessary to retain the enone or dienone structure of curcumin in order to retain activity. Other phenolic analogues in series 1 included analogue 2g, where the methoxy groups of curcumin have been removed, and 2i, which is an isomer of curcumin. Active analogues in series 2 (9r, 9t and 12a) also retained phenolic groups, although not all phenolic analogues were active including 9a, 9s and 9y. Active analogues in series 3 (8a, 8t, 17f, 17g and 17h) retain phenolic groups.

Most interesting is the activity of analogues that do not retain phenolic groups. Two analogues in series 1 (6b and 7b) are dienones, similar to curcumin. However, both 6b and 7b are devoid of ring substituents but contain a single alkyl group attached to the central methylene carbon. By comparison, analogue 2b, which has no ring substituents or an alkyl group attached to the central methylene carbon, and analogues 4b and 5b, which are similar to 6b and 7b but with dialkylation of the central methylene, are inactive. An explanation of these properties is shown in FIG. 3. Curcumin (2a, FIG. 3, top) has been proposed to form the stable phenoxy radical in radical trapping reactions either through direct abstraction of the phenolic hydrogen, or by way of initial ionization of an acidic proton from the central methylene, followed by electron transfer to form a carbon-centered radical that can isomerize to the phenoxy radical.⁹ The pathway is dictated by reaction conditions. In the case of analogues 6b and 7b (FIG. 3, bottom), stabilized tertiary carbon-centered radicals can form in the reaction of 6b or 7b with ABTS in the TRAP assay. This is not possible with the dialkylated analogues 4b and 5b. Analogue 2b likely is inactive because formation of a secondary carbon-centered radical is less favored than formation of tertiary radicals.

The FRAP assay measures the ability of a compound to reduce the ferric tripyridyltriazine complex to the colored ferrous complex. The results of the FRAP assay of anti-oxidant activities of curcumin and analogues are shown in FIG. 4. The results show similarities as well as differences compared to the TRAP assay. In series 1, curcumin (2a) is most active, and other phenolic analogues including 3a, 2g and 2i are active. Likewise, in series 2 and 3, active analogues 12a, 8a, 17h and 17f are phenolic compounds that also were active in the TRAP assay. Analogue 7b, which is devoid of phenolic groups but contains a benzyl group attached to the central methylene of the curcumin basic structure and was active in the TRAP assay, is also active in the FRAP assay whereas the related 6b was active only in the TRAP assay. Especially interesting are the results with analogues 2h and 9l, which are devoid of phenolic groups. Analogue 2h which is comparable to curcumin in the FRAP assay, contains dimethylamino groups in place of phenolic groups in the basic curcumin structure and contains no other functional groups. This raises the possibility of developing analogues that are more active than curcumin. The mechanism of the anti-oxidant activities of 7b and 2h in the FRAP assay may involve formation of carbon-centered radicals, however this remains to be investigated.

Selected analogues of curcumin that are devoid of phenolic groups were active in both the TRAP assay and the FRAP assay, and that some of these are active based upon their abilities to form stable carbon-centered radicals. Other analogues that are devoid of phenolic groups also exhibit activity by mechanisms that must still be determined. Most of the active analogues of curcumin, however, are able to form phenoxy radicals, and this is likely the basis of their anti-oxidant activities. With this set of analogues, we now have insight into the role of anti-oxidant activity in the multiple biological activities reported for curcumin. As a result, it is found that these are useful analogues for use the treatment process.

What is claimed is:

1. A method for treatment of cancer in mammals by suppression of NFκB expression comprising:
providing a therapeutically effective amount of a curcumin derivative administering the curcumin derivative to the mammal.

2. The method of claim of claim 1, wherein the composition further comprises a pharmaceutically acceptable carrier.

3. The method of claim 1, wherein administering comprises administering by a method of administration selected from the group consisting of oral administration, parenteral administration, transcutaneous administration, intranasal administration, intramuscular administration and rectal administration.

4. The method of claim 1 wherein the suppression of NFκB is direct suppression.

5. The method of claim 1 wherein the suppression of NFκB is indirect suppression of at least one member of the following group consisting of IKK, SFK kinases, other upstream kinases.
